(19) United States
(12) Patent Application Publication
(10) Pub. No.: US 2006/0205727 A1
(43) Pub. Date: Sep. 14, 2006

(54) Combination therapy for endothelial dysfunction, angina and diabetes

(76) Inventor: Wayne Kaesemeyer, Augusta, GA (US)

Correspondence Address: PATREA L. PABST;PABST PATENT GROUP LLP, 400 COLONY SQUARE, SUITE 1200, ATLANTA, GA, 30361, US

(21) Appl. No.: 11/373,658

(22) Filed: Mar. 10, 2006

Related U.S. Application Data
(60) Provisional application No. 60/660,625, filed on Mar. 11, 2005.
(60) Provisional application No. 60/675,118, filed on Apr. 27, 2005.

(51) Int. Cl. A61K 31/537; A61K 31/401; A61K 31/366; A61K 31/22

(52) U.S. Cl. 514/237.5; 514/423; 514/460; 514/548

(57) ABSTRACT

The combination of a HMG CoA reductase inhibitor like a statin, such as simvastatin, with a pFox inhibitor such as trimetazidine (“Simetazidine”) is particularly advantageous for treatment of end-stage complications, such as acute coronary syndrome (ACS) and chronic angina, especially in type II diabetics. The combination therapy is also useful in the treatment and/or prevention of chronic heart failure (CHF) and peripheral arterial disease (PAD). The combination of a nitric oxide (NO) mechanism with increased NO production with pFox inhibition simultaneously treats both the effect and the cause of angina. One or more oral hypoglycemic compounds (biguanides, insulin sensitizers, such as thiazolidinediones, α-glucosidase inhibitors, insulin secretagogues, and dipeptidyl peptidase IV inhibitors), protein kinase C (PKC) inhibitors, and acetyl-CoA carboxylase inhibitors can also be used in combination with the HMG CoA reductase inhibitors and/or pFox inhibitors, especially in type II diabetics, to control glucose levels and treat endothelial dysfunction. The drugs can be given in combination (e.g. a single tablet) or in separate dosage forms, administered simultaneously or sequentially. In the preferred form the statin is given in a dose of between 5 and 80 mg/day in two separate doses, and the pFox inhibitor is administered in a sustained or extended dosage formulation at a dose of 20 mg three times a day or 35 mg two times a day. The dose of the oral hypoglycemic, PKC inhibitor, or acetyl-CoA carboxylase inhibitor varies with the type of drug used.

### CROSS-REFERENCE TO RELATED APPLICATIONS

This application claims benefit under 35 U.S.C. 119 to U.S. Provisional Application No. 60/660,625 entitled “Combination pFox-HMG CoA Reductase Inhibitor Therapy” filed Mar. 11, 2005 by Wayne Kaesemeyer and U.S. Provisional Application No. 60/675,118 entitled “Combination Therapy for Endothelial Dysfunction and Diabetes” filed Apr. 27, 2005 by Wayne Kaesemeyer.

### FIELD OF THE INVENTION

The present invention is generally in the field of treating endothelial dysfunction, angina and diabetes, especially through the use of a combination of a partial fatty acid oxidation (“pFox”) inhibitor, such as trimetazidine, an HMG CoA reductase inhibitor (“statin”), one or more oral hypoglycemic compounds, protein kinase C inhibitors, and acetyl-CoA carboxylase inhibitors.

### BACKGROUND OF THE INVENTION

Atherosclerosis of the coronary and peripheral vasculature is the leading cause of death from cardiovascular disease worldwide. Cholesterol deposition in the arterial wall is central to the pathogenesis. Hypertension, cigarette smoking, left ventricular hypertrophy, obesity and family history of premature coronary heart disease have been identified as other independent risk factors for development of coronary vascular disease.

The 3-hydroxy-3-methylglutaryl-coenzyme A (“HMG-CoA) reductase inhibitors, or “statins”, have revolutionized treatment of high cholesterol. Studies have demonstrated that most of the statins reduce the risk of major coronary events by 30% and produce a greater absolute benefit in patients with higher baseline risk. Statins have many activities besides directly lowering cholesterol, many of which are still poorly understood, but most of which involve upregulation of endothelium-derived nitric oxide production. Almost one third of patients in primary and secondary prevention programs treated with cholesterol-lowering agents fail to reach target LDL levels. There is also a residual cardiovascular morbidity observed in clinical trials in spite of statin treatment. See Catianeo, et al., Expert Opin. 14(11):1559-1586 (2004). Accordingly, there remains a need for new and more effective agents.

A large proportion of patients have both hypercholesterolemia and hypertension, two well-known, and possibly related, risk factors associated with increased coronary vascular disease (Id.) Preliminary studies have indicated that statins may decrease blood pressure, regardless of whether or not patients were on antihypertensive therapy with angiotensin-converting enzyme (ACE) inhibitors and calcium channel blockers. (Id.) The effect is believed to relate to the effect of the statins on endothelial function and/or the rennin-angiotensin system. There is some evidence that treatment with statins may enhance endothelial nitric oxide synthase activity and decrease myocardial infarct size.

Syrkin, et a., Kardiologia 7:49-52 (2003) describes treatment of patients with angina and claudication by administering trimetazidine to a group of patients being treated with simvastatin and plavix. In the trimetazidine group there was 46.7% improvement in angina and 33.8% improvement in claudication, both of which were significant as compared to the control, patients receiving simvastatin and plavix and treated with placebo.

Diabetes has also been identified as a major risk factor for development of coronary vascular disease. Diabetes refers to a disease process derived from multiple causative factors and characterized by elevated levels of plasma glucose or hyperglycemia. Although diabetes has been primarily regarded as a disorder of glucose metabolism and homeostasis, it has more recently been viewed as a constellation of metabolic disturbances, including abnormalities of carbohydrate metabolism, adipose storage, lipid metabolism, and protein biochemistry. Diabetes has been commonly characterized as a disease of impaired skeletal muscle glucose uptake, however, diabetes also adversely affects hepatic, muscle, adipose, and vascular function. It is this last effect that may represent the greatest mortality hazard. Diabetes creates an environment adverse to vascular function through a wide variety of dysmetabolic assaults.

It is therefore an object of the present invention to provide formulations that are useful in treating angina, myocardial infarction, atherosclerosis and other disorders involving endothelial dysfunction.

It is also an object of the present invention to provide formulations for the treatment of diabetes, which leads to disorders involving endothelial dysfunction.

### BRIEF SUMMARY OF THE INVENTION

The combination of a HMG CoA reductase inhibitor like a statin, such as simvastatin, with a pFox inhibitor such as trimetazidine (“Simetazidine”), is particularly advantageous for treatment of end-stage complications, such as acute coronary syndrome (ACS) and chronic angina, especially in type II diabetics. The combination therapy is also useful in the treatment and/or prevention of chronic heart failure (CHF) and peripheral arterial disease (PAD). A nitric oxide agonist, nitric oxide generator or an upregulator of nitric oxide synthase can also be administered or a pFOX inhibitor or HMG CoA reductase inhibitor having such an activity can also be administered. The combination of a nitric oxide (NO) mechanism that results in increased NO production with pFox inhibition simultaneously treats both the effect and the cause of angina. One or more oral hypoglycemic compounds such as biguanides, insulin sensitizers, such as thiazolidinediones, α-glucosidase inhibitors, insulin secretagogues, and dipeptidyl peptidase IV inhibitors, protein kinase C (PKC) inhibitors, and acetyl-CoA carboxylase inhibitors can also be used in combination with the HMG CoA reductase inhibitors and/or pFox inhibitors, especially in type II diabetics, to control glucose levels and treat endothelial dysfunction. The drugs can be given in combination (e.g. a single tablet) or in separate dosage forms, administered simultaneously or sequentially. In the preferred form the statin is given in a dose of between 5 and 80 mg/day in two separate doses, and the pFox inhibitor is administered in a sustained or extended dosage formulation at a dose of 20 mg three times a day or 35 mg two times a day. The dose of the oral hypoglycemic, PKC inhibitor, or acetyl-CoA carboxylase inhibitor varies with the type of drug used.

### BRIEF DESCRIPTION OF THE DRAWING

FIG. 1 is a diagram of the prior art treatments versus the treatments described herein, graphed based on degree of invasiveness. PCI is percutaneous coronary intervention; CABG is coronary artery bypass grafting; HR is heart rate; MVO₂ is myocardial oxygen consumption; QT_{c} is the EKG QT interval corrected for heart rate; FFA is free fatty acid; ATP is adenosine triphospate; HDL is high density lipoprotein; and CRP is C-reactive protein.

### DETAILED DESCRIPTION OF THE INVENTION

A combination therapy has been designed to provide the benefits of treatment with a trimetazidine or other pFox inhibitor in combination with an HMG CoA reductase inhibitor, such as a statin. One or more oral hypoglycemics, including biguanides, insulin sensitizers, α-glucosidase inhibitors, insulin secretagogues, may also be used in combination with the HMG CoA reductase inhibitor and pFox inhibitor for the treatment of diabetes and endothelial dysfunction. In addition, dipeptidyl peptidase IV inhibitors, which are also hypoglycemics, protein kinase C inhibitors, acetyl-CoA carboxylase inhibitors, or selective rho-kinase inhibitors may be used in combination with the HMG CoA reductase inhibitor and/or pFox inhibitor.

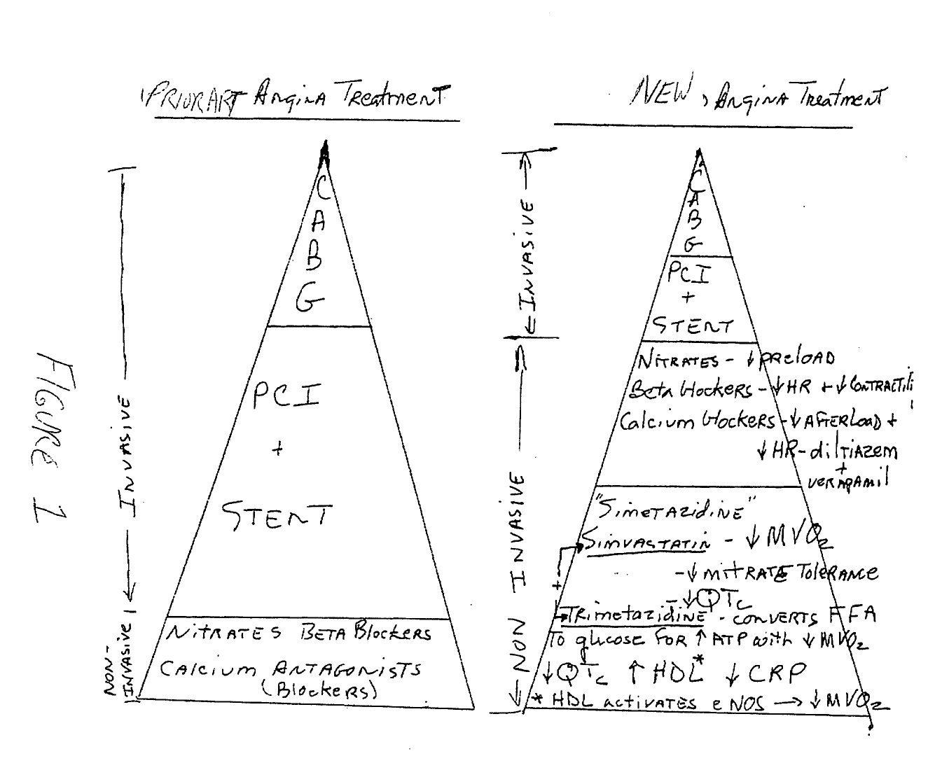

As shown in FIG. 1, the prior art treatments are either invasive, CABG or PCI and stent, or the combination of nitrates, beta blockers, and calcium antagonists (blockers). The treatments described herein provide many more non-invasive options, which are less invasive than the prior art treatments. The statin decreases myocardial oxygen consumption while decreasing nitrate tolerance and QT_{c}. The trimetazidine or other pFOX inhibitor alters substrate utilization from free fatty acids to glucose, resulting in an increase in ATP with a decrease in myocardial oxygen consumption. This decreases QT_{c}, increases HDL (which activates eNOS), thereby further decreasing myocardial oxygen consumption.

### I. FORMULATIONS

The formulations can consist of the drugs in a single formulation, or in a package providing two or more drugs. The pharmaceutically effective doses for the statins, pFox inhibitors, dipeptidyl peptidase IV inhibitors, protein kinase C inhibitors, acetyl-CoA carboxylase inhibitors, and selective rho-kinase inhibitors are those sufficient to provide a desirable diminution in the risk or prevalence of cardiovascular disease and/or diabetes. Most preferably, the effective amount will be one which lowers the recipient's whole blood or serum cholesterol levels, particularly LDL levels, or maintains those levels within a concentration range reasonable for the individual in question, taking into account his or her initial levels, overall health, family history for cardiovascular maladies, age, weight, etc. while at the same time increasing oxygen flow (reducing ischemia) and relieving angina. The pharmaceutically effective doses for the oral hypoglycemics are those sufficient to provide adequate blood glucose control in diabetics.

### A. pFox Inhibitors

A “pFox inhibitor” is any compound that shifts myocardial substrate utilization from free fatty acid to glucose, regardless of the enzyme inhibited. A pFox inhibitor, most preferably one which does not prolong QT intervals, can be used in combination with a HMG CoA reductase inhibitor, common referred to as “statins”, and optionally an oral hypoglycemic for the treatment of endothelial dysfunction and diabetes. The combination of a pFox inhibitor with an HMG CoA reductase inhibitor has a dual mechanism of both reversing endothelial dysfunction through the nitric oxide pathway and reducing ischemia thereby relieving angina and improving long term outcome.

The piperazine derivatives ranolazine and trimetazidine are examples of pFox inhibitors whose mechanism of action involves shifting ATP production away from fatty acid oxidation in favor of glucose oxidation. Inhibition of fatty acid oxidation results in a reduction in the inhibition of pyruvate dehydrogenase and an increase in glucose oxidation. The amount of oxygen required to phosphorylate a given amount of ATP is greater during fatty acid oxidation than during carbohydrate oxidation. Thus, increasing glucose oxidation reduces oxygen demand without decreasing the ability of tissue to do work. Trimetazidine has also been shown to: (1) reduce the levels of plasma C-reactive protein in the course of acute myocardial infarction treated with streptokinase and intravenous trimetazidine infusion (Blaha et al., Acta Medica, 44(4), 135-40 (2001); (2) have a beneficial effect in patients with circulatory deficiency through the improvement of hemostatic and biochemical parameters (Demidova et al., Ter. Arkh., 70(6), 41-44 (1998); and (3) induce functional improvement in patients with dilated cardiomyopathy via significant improvement of left ventricular function (Barsotti et al., Heart, 91(2), 161-165 (2005). Clinical results also suggest that the inflammatory response was limited in patients treated with trimetzidine (Barostti et al.). The structures of ranolazine and trimetazidine are shown below:

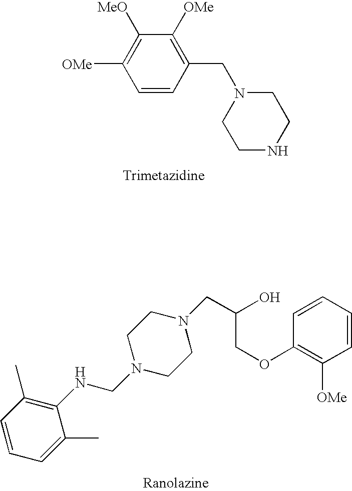

Ranolazine and trimetazidine are described in U.S. Pat. Nos. 4,567,264, and 4,663,325, respectively. Ranolazine is not preferred because it causes QT interval prolongation and undergoes metabolism via the CYP3A4 system in the liver and is prone to drug-drug interactions which further aggravate QT interval prolongation. Other suitable pFOX inhibitors include perhexiline maleate and mildronate. The structure of perhexiline maleate and mildronate are shown below.

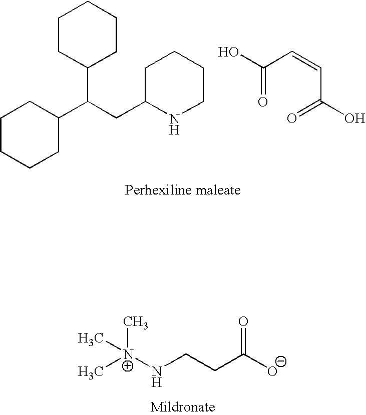

Perhexiline maleate is an anti-anginal agent. Its mechanism of action as an anti-anginal agent has not been fully elucidated in humans; however, in vitro studies suggest that perhexiline causes inhibition of myocardial fatty acid catabolism (e.g. by inhibition of carnitine palmitoyltransferase-1: CPT-1) with a concomitant increase in glucose utilization and consequent oxygen-sparing effect. This is likely to have two consequences:

(i) increased myocardial efficiency, and

(ii) decreased potential for impairment of myocardial function during ischemia.

The inhibition of CPT-1 is likely to contribute to the anti-ischaemic effects of perhexiline. Animal studies indicate a direct action of the medicine on the myocardium dependent in part on the marked degree of tissue binding. In vitro studies indicate a non-specific depressant effect of perhexiline on all smooth muscle. It also inhibits the spontaneous depolarisation of Purkinje fibres in the dog myocardium and reduces sodium and potassium conductance. The dosage range of perhexiline is typically 100 mg to 300 mg daily; however, dosages of 400 mg per day may be required. Perhexiline maleate is commercially available in 100 mg tablets.

Mildronate ameliorates cardiac function during ischemia by modulating myocardial energy metabolism. Biochemical and pharmacological evidence suggests that the mechanism of action of mildronate is based on the regulatory effect on carnitine concentration, whereby mildronate treatment shifts the myocardial energy metabolism from fatty acid oxidation to the more favorable glucose oxidation under ischemic conditions (Dambrova et al. Trends in Cardiovascular Medicine, Vol. 12, No. 6 (2002)). The dosage range for mildronate is typically between 500 mg and 1000 mg daily, in divided doses. Mildronate is commercially available in 250 mg and 500 mg capsules as well as a 10% injectable solution and a syrup.

### B. Statins

There are a number of statins that are available and approved for use. These include mevastatin, lovastatin, pravastatin, simvastatin, velostatin, dihydrocompactin, fluvastatin, atorvastatin, dalvastatin, carvastatin, crilvastatin, bevastatin, cefvastatin, rosuvastatin, pitavastatin, and glenvastatin. The preferred statins include pravastatin, torvastain, fluvastatin, lovastatin, and metastatin. The statin compounds are administered in regimens and at dosages known in the art. For instance, Cervistatin, which is sold by Bayer Corporation as Baycol™, has a recommended dosage of 0.3 mg once daily in the evening, with a starting dose for patients with significant renal failure of 0.2 mg per day, taken once daily in the evening. Fluvastatin sodium, marketed by Novartis Pharmaceuticals as Lescol™, is recommended for a 20-80 mg daily oral dose range, preferably between 20 and 40 mg/day for the majority of patients. 20 to 40 mg daily doses are preferably taken once daily at bedtime. 80 mg daily doses is prescribed as 40 mg doses b.i.d. and recommended only for those individuals in which the 40 mg daily dose is inadequate to lower LDL levels satisfactorily. Atorvastatin, offered by Parke Davis as Lipitor™, has a recommended starting daily dose of 10 mg once daily, with an overall daily dose range of from 10 to 80 mg. Simvastatin, marketed by Merck & Co., Inc., may be administered with a starting dose of 20 mg once a day in the evening, or a 10 mg dose per day for those requiring only a moderate reduction in LDL levels. The recommended overall daily dosage range taken as a single evening dose is from 5 to 80 mg. Pravastatin sodium, sold as Pravachol™ by Bristol-Meyers Squibb, has a recommended starting dose of 10 or 20 mg per day, taken daily as a single dose at bedtime, with a final overall daily range of from 10 to 40 mg. Lovastatin, sold by Merck & Co. as Mevacor™, has a recommended daily starting dosage of 20 mg per day taken with the evening meal. The recommended final daily dosage range is from 10 to 80 mg per day in single or divided doses.

HMG CoA reductase inhibitors have been shown to lower blood cholesterol levels by upregulating lipoprotein clearance receptors in the liver (Brown and Goldstein, (1986) Science 232, 34-47). Based on the Heart Protection Study and the A to Z trial the preferred simvastatin dose should be 40 mg total/day. This could be formulated, for example, as 20 mg simvastatin immediate release combined with 35 mg of the new trimetazidine MR for BID dosing or it could be 13.33 mg simvastatin/20 mg immediate release trimetazidine for TID dosing. In April 2004, the U S Food and Drug administration approved the use of simvastatin for treating existing coronary heart disease and diabetes irrespective of cholesterol levels. This was based on the results of the Heart Protection Study, a seven year, 22,000 patient study which showed benefits regardless of the levels of cholesterol of the individuals in the trial. In the PROVE-IT trial some benefits were seen in the treatment of acute coronary syndrome in the first 30 days of the trial with 80 mg/day of atorvastatin that was believed to be unrelated to cholesterol lowering.

### C. Nitric Oxide Agonists/Generators/Upregulators of Nitric Oxide Synthase

In one embodiment, a nitric oxide agonist, nitric oxide generator or an upregulator of nitric oxide synthase is given in combination with an HMG CoA reductase inhibitor and a partial fatty acid oxidation (“pFox”) inhibitor. Suitable nitric oxide agonists or upregulators of nitric oxide synthase include angiotensin II receptor blockers (ARB's), angiotensin converting enzyme (ACE) inhibitors, endothelial nitric oxide synthase agonists, peroxisome proliferator-activated receptor activators, and cilostazol.

Angiotensin-II receptor antagonists (or blockers) are selective for the angiotensin II (type 1 receptor). Examples of angiotensin-II receptor antagonists are losartan (Cozaar) (50-200 mg/day), valsartan (Diovan) (80 to 320 mg), irbesartan (Avapro) (75-300 mg/day), candesartan (Atacand) (8-64 mg/day) and telmisartan (Micardis) (40-160 mg/day). Other angiotensin-II receptor antagonists currently under investigation include eprosartan, tasosartan and zolarsartan.

Angiotensin Converting Enzyme (ACE) Inhibitors generate nitric oxide in the wall of small arteries. Suitable angiotensin-converting enzyme inhibitors along with recommended daily doses, include, but are not limited to, alacepril, benazepril (10-80 mg/day), captopril (25-450 mg/day), ceranapril, cilazapril, delapril, duinapril, enalapril (5-40 mg/day), enalaprilat, fosinopril (10-80 mg/day), imidapril, lisinopril (10-40 mg/day), moexipril (7.5-30 mg/day), moveltipril, pentopril, perindopril (4-16 mg/day), quinapril (10-80 mg/day), ramipril (2.5-20 mg/day), rentipril, spirapril, temocapril, trandolapril (1-8 mg/day), and zofenopril. The angiotensin-converting enzyme inhibitors are described more fully in the literature, such as in Goodman and Gilman, The Pharmacological Basis of Therapeutics (9th Edition), McGraw-Hill, 1995; and the Merck Index on CD-ROM, Twelfth Edition

There are a number of compounds that are known to upregulate eNOS expression and/or increase eNOS activity. These are described in U.S. Patent Publication No. 20040254238 and U.S. Pat. No. 6,425,881, and include acetylcholine, cyclosporin A, FK506, felodipine, nicorandil, nifedipine, diltiazem, resveritrol, sapogrelate, quinapril and nebivolol. The combination of nebivolol and a pFox inhibitor, such as trimetazadine, should be beneficial for the treatment of angina and hypertension. Statins are also known activators of eNOS. For example, high density lipoprotein (“HDL”) causes potent stimulation of eNOS activity through binding to SR-BI. Statins, such as simvastatin and atorvastatin increase the concentration of HDL (atorvastatin more so than simvastatin). Mixtures of NO donors may also have this effect as described in U.S. Pat. No. 5,543,430 which describes nitroglycerin as an eNOS agonist in combination with arginine.

In humans, peroxisome proliferator-activated receptors (PPARs) are found in key target tissues for insulin action such as adipose tissue, skeletal muscle, and liver. Activation of PPARγ nuclear receptors regulates the transcription of insulin-responsive genes involved in the control of glucose production, transport, and utilization. In addition, PPARγ-responsive genes also participate in the regulation of fatty acid metabolism. Suitable peroxisome proliferator-activated receptor activators include those agents that bind to the peroxisome proliferator-activated receptor gamma (PPAR-γ). Examples of such compounds include the thiazolidinediones, troglitazone (Rezulin), rosiglitazone (Avandia) and pioglitazone (Actos), which are described below.

Cilostazol (6-[4-(1-cyclohexyl-1H-tetrazol-5-yl)butoxy]-3,4-dihydro-2(1H)-quinolinone, a treatment for intermittent claudication, is sold as PLETAL™ Otsuka America Pharmaceutical. Intermittent claudication is a condition caused by narrowing of the arteries that supply the legs with blood. Patients with intermittent claudication develop pain when they walk because not enough oxygen-containing blood reaches the active leg muscles. Cilostazol reduces the pain of intermittent claudication by dilating the arteries, thereby improving the flow of blood and oxygen to the legs. Cilostazol and some of its metabolites are cyclic AMP (cAMP) phosphodiesterase III inhibitors (PDE III inhibitors), inhibiting phosphodiesterase activity and suppressing cAMP degradation with a resultant increase in cAMP in platelets and blood vessels, leading to inhibition of platelet aggregation and vasodilation. Cilostazol reversibly inhibits platelet aggregation induced by a variety of stimuli, including thrombin, ADP, collagen, arachidonic acid, epinephrine, and shear stress. The drug is routinely used at doses of 100-200 mg/day.

### D. Oral Hypoglycemic Compounds

One or more oral hypoglycemic compounds, including a biguanide, thiazolidinedione, alpha-glucosidase inhibitor, insulin secretagogue, dipeptidyl peptidase IV inhibitor, or protein kinase C inhibitor can be used in combination with a pFox inhibitor and/or an HMG CoA reductase inhibitor for the treatment of endothelial dysfunction and diabetes.

### 1. Biguanides

The biguanides that can be used include metformin and phenformin. These compounds have been well described in the art, e.g. in U.S. Pat. No. 6,693,094. Metformin (N,N-dimethylimidodicarbonimidicdiamide; 1,1-dimethylbiguanide; N,N-dimethylbiguanide; N,N-dimethyldiguanide; N′-dimethylguanylguanidine) is an anti-diabetic agent that acts by reducing glucose production by the liver and by decreasing intestinal absorption of glucose. It is also believed to improve the insulin sensitivity of tissues elsewhere in the body (increases peripheral glucose uptake and utilization). Metformin improves glucose tolerance in impaired glucose tolerant (IGT) subjects and Type 2 diabetic subjects, lowering both pre- and post-prandial plasma glucose. Metformin is generally not effective in the absence of insulin. Bailey, Diabetes Care 15:755-72 (1992). Metformin (Glucophage™) is commonly administered as metformin HCl. Metformin is also available in an extended release formulation (Glucophage XR™). Dose ranges of metformin are between 10 to 2550 mg per day, and preferably 250 to 2000 mg per day.

### 2. Insulin Sensitizers

### a. Thiazolidinediones

Thiazolidinediones that can be used include troglitazone (Rezulin™), rosiglitazone (sold as Avandia™ by GlazoSmithKline), pioglitazone (sold as Actos™ by Takeda Pharmaceuticals North America, Inc. and Eli Lilly and Company), ciglitazone, englitazone, R483 (produced by Roche, Inc.) and pioglitazone.

Such compounds are well-known, e.g., as described in U.S. Pat. Nos. 5,223,522, 5,132,317, 5,120,754, 5,061,717, 4,897,405, 4,873,255, 4,687,777, 4,572,912, 4,287,200, and 5,002,953; and Current Pharmaceutical Design 2:85-101 (1996). The thiazolidinediones work by enhancing insulin sensitivity in both muscle and adipose tissue and to a lesser extent by inhibiting hepatic glucose production. Thiazolidinediones mediate this action by binding and activating peroxisome proliferator-activated receptor-gamma (PPARγ). Effective doses include troglitazone (10-800 mg/day), rosiglitazone (1-20 mg/day), and pioglitazone (15-45 mg/day). Phase II studies with the glitazone; R483, have been completed and show a significant dose-dependent reduction of HbA1 c. R483 has been tested at doses of 5-40 mg/day.

### 3. Alpha-Glucosidase Inhibitors

Alpha-glucosidase inhibitors competitively inhibit alpha-glucosidase, which metabolizes carbohydrates, thereby delaying carbohydrate absorption and attenuating post-prandial hyperglycemia. Clissod et al., Drugs 35:214-23 (1988). This decrease in glucose allows the production of insulin to be more regular, and as a result, serum concentrations of insulin are decreased as are HbA1 c levels.

A variety of glucosidase inhibitors are known to one of ordinary skill in the art and described in U.S. Pat. Nos. 6,821,977 and 6,699,904. Preferred glucosidase inhibitors include acarbose, adiposine, voglibose, miglitol, emiglitate, camiglibose, tendamistate, trestatin, pradimicin-Q and salbostatin. The glucosidase inhibitor, acarbose, and the various amino sugar derivatives related thereto are described in U.S. Pat. Nos. 4,062,950 and 4,174,439 respectively. The glucosidase inhibitor, adiposine, is described in U.S. Pat. No. 4,254,256. The glucosidase inhibitor, voglibose, 3,4-dideoxy-4-[[2-hydroxy-1-(hydroxymethyl)ethyl]amino]-2-C-(hydroxymethyl )-D-epi-inositol, and the various N-substituted pseudo-aminosugars related thereto, are described in U.S. Pat. No. 4,701,559. The glucosidase inhibitor, miglitol, (2R,3R,4R,5S)-1-(2-hydroxyethyl)-2-(hydroxymethyl)-3,4,5-piperidinetriol, and the various 3,4,5-trihydroxypiperidines related thereto, are described in U.S. Pat. No. 4,639,436. The glucosidase inhibitor, emiglitate, ethyl p-[2-[(2R,3R,4R,5S)-3,4,5-trihydroxy-2-(hydroxymethyl)piperidino]lethoxy]-benzoate, the various derivatives related thereto and pharmaceutically acceptable acid addition salts thereof, are described in U.S. Pat. No. 5,192,772. The glucosidase inhibitor, MDL-25637, 2,6-dideoxy-7-O-.beta.-D-glucopyrano-syl-2,6-imino-D-glycero-L-gluco-heptitol, the various homodisaccharides related thereto and the pharmaceutically acceptable acid addition salts thereof, are described in U.S. Pat. No. 4,634,765. The glucosidase inhibitor, camiglibose, methyl 6-deoxy-6-[(2R,3R,4R,5S)-3,4,5-trihydroxy-2-(hydroxymethyl)piperidino]-.al pha.-D-glucopyranoside sesquihydrate, the deoxy-nojirimycin derivatives related thereto, the various pharmaceutically acceptable salts thereof and synthetic methods for the preparation thereof, are described in U.S. Pat. Nos. 5,157,116 and 5,504,078. The glucosidase inhibitor, salbostatin and the various pseudosaccharides related thereto, are described in U.S. Pat. No. 5,091,524. The daily dose of alpha-glucosidase inhibitors is usually 0.1 to 400 mg, and preferably 0.6 to 300 mg. Effective dosages of both acarbose and miglitol are in the range of about 25 up to about 300 mg/day.

### 4. Insulin Secretagogues

### a. Sulfonylureas

Sulfonylureas are a class of compounds that are well-known in the art, e.g., as described in U.S. Pat. Nos. 3,454,635, 3,669,966, 2,968,158, 3,501,495, 3,708,486, 3,668,215, 3,654,357, and 3,097,242. These compounds generally operate by lowering plasma glucose by increasing the release of insulin from the pancreas. Their action is initiated by binding to and closing a specific sulfonylurea receptor (an ATP-sensitive K⁺ channel) on pancreatic beta-cells. This closure decreases K⁺ influx, leading to depolarization of the membrane and activation of a voltage-dependent Ca²⁺ channel. The resulting increased Ca²⁺ flux into the beta-cell, activates a cytoskeletal system that causes translocation of insulin to the cell surface and its extrusion by exocytosis.

Examples of sulfonylureas (with typical daily dosages indicated in parentheses) include acetohexamide (in the range of about 250 up to about 1500 mg), chlorpropamide (in the range of about 100 up to about 500 mg), tolazimide (in the range of about 100 up to about 1000 mg), tolbutamide (in the range of about 500 up to about 3000 mg), gliclazide (in the range of about 80 up to about 320 mg), glipizide (Glucotrol™) (in the range of about 5 up to about 40 mg), glipizide gastrointestinal therapeutic system (GITS) (extended release) (Glucotrol™) (in the range of about 5 up to about 20 mg), glyburide (in the range of about 1 up to about 20 mg), micronized glyburide (in the range of about 0.75 up to about 12 mg), glimepiride (in the range of about 0.5 up to about 8 mg), and AG-EE 623 ZW. In a preferred embodiment, the sulfonylurea is glimepiride in a daily dose range of 0.5 to 4 mg.

### b. Non-Sulfonylureas

Suitable non-sulfonylureas are described in U.S. Pat. Nos. 6,652,838, 6,734,175, and 6,830,759, and include D-phenylalanine derivatives, such as nateglinide (N-[[4-(1-methylethyl)cyclohexyl]carbonyl]-D-phenylalanine) and meglitinides, such as repaglinide. Nateglinide is a fast-acting antidiabetic agent which functions to stimulate insulin production. Meglitinides, are non-sulfonylurea hypoglycemic agents that have insulin secretory capacity. For example, repaglinide appears to bind to ATP-sensitive potassium channels on pancreatic beta cells and thereby increases insulin secretion. For repaglinide, the effective daily dosage may be in the range of about 0.5 mg up to about 16 mg.

### 5. Dipeptidyl Peptidase IV Inhibitors

Dipeptidyl peptidase-IV (DPP-IV) inhibitors are potential drugs for the treatment of type 2 diabetes. The original concept that inhibition of DPP-IV would improve glucose tolerance was based on the observation that glucagon-like peptide-1 (GLP-1) is rapidly cleaved and inactivated by the protease DPP-IV (Holst J J and Deacon C F. Diabetes 47:1663-1670 (1998)). Inhibition of this proteolytic inactivation should prolong the action of GLP-1, which is released postprandially from the L-cells in the gut and increases insulin secretion (the ‘incretin’ concept), resulting in improved glucose tolerance. GLP-1 has also been shown to reduce postprandial and fasting glycemia in subjects with type 1 and type 2 diabetes (Ahren B. BioEssays 20:642-651 (1998))

The potential of using this approach in the treatment of diabetes is illustrated in studies showing that DPP IV-deficient mice (Marguet et al. Proc Natl Acad Sci USA 97:6874-6879 (2000)) and rats (Nagakura T et al. Biochem Biophys Res Commun 284:501-506 (2001)) exhibit increased insulin secretion and glucose tolerance. Furthermore, in diabetic animal models, improved glucose tolerance and insulin response to oral glucose have been demonstrated by several different DPP IV inhibitors (Pederson et al. Diabetes 47:1253-12581(1998)).

In another embodiment, DPP-IV inhibitors are used in combination with an HMG CoA reductase inhibitor and/or a pFox inhbitor for the treatment of patients with diabetes or metabolic syndrome and endothelial dysfunction. Suitable DPP IV inhibitors include those compounds described in U.S. Pat. Nos. 6,683,080, 6,861,440, 6,500,804, and U.S. Patent Publication No. 20040224875, including L-threo-isoleucyl pyrrolidide, L-allo-isoleucyl thiazolidide, L-allo-isoleucyl pyrrolidide; and salts thereof or valine pyrrolidide, NVP-DPP728A (1-[[[2-[{5-cyanopyridin-2-yl}amino]ethyl]amino]-acetyl]-2-cyano-(S)-pyrrolidine) LAF-237 (1-[(3-hydroxy-adamant-1-ylamino)-acetyl]-pyrrolidine-2(S)-carbonitrile); TSL-225 (tryptophyl-1,2,3,4-tetra-hydroisoquinoline-3-carboxylic acid), FE-999011 ([(2S)-1-([2′S]-2′-amino-3-′,3′dimethyl-butanoyl)-pyrrolidine-2-carbonitrile]), GW-229A, 815541, MK-431 or PT-100 (Point Therapeutics). The DP-14 inhibitors may be given at a dosage of from about 0.1-300 mg/kg per day (preferred 1-50 mg/kg per day). Preferred daily doses for NVP DPP728 are 100-300 mg/day.

### 6. Combination of Oral Hypoglycemics

In another embodiment, more than one oral hypoglycemic compound is used in combination with a pFox inhibitor and HMG CoA reductase inhibitor. Several of the available oral hypoglycemic agents have been studied in combination and have been shown to further improve glycemic control when compared to monotherapy (Riddle M. Am J Med 108(suppl 6a): 15S-22S (2000)). As with monotherapy, the choice of a second agent should be based on individual characteristics. Reasonable combinations of agents include a sulfonylurea plus metformin, a sulfonylurea plus an alpha-glucosidase inhibitor, a sulfonylurea plus a thiazolidinedione, metformin plus repaglinide, biguanide plus alpha-glucosidase inhibitor, metformin plus a thiazolidinedione, thiazolidinedione plus DP IV inhibitor, and metformin plus DP IV inhibitor. For example, an oral medication containing metformin plus rosiglitazone is sold as Avandamet™ by GlaxoSmithKline, Inc (in a preferred dose range of from 1 mg/day rosiglitazone/250 mg/day metformin to 8 mg/day rosiglitazone/2,000 mg/day metformin. Oral medications combining glyburide and metformin (Glucovance™) (in a preferred dose range of from 1.25 mg/day glyburide/250 mg/day metformin to 10 mg/day glyburide/2,000 mg/day metformin) and glipizide and metformin (Metaglip™) (in a preferred dose range of from 2.5 mg/day glipazide/250 mg/day metformin to 10 mg/day glipazide/2,000 mg/day metformin) are sold by Bristol Myers Squibb.

In some cases, three oral hypoglycemic compounds, such as sulfonylurea, metformin, thiazolidinedione or sulfonylurea, metformin, alpha-glucosidase inhibitor, may be combined.

### E. Protein Kinase C (PKC) Inhibitors

Recent studies have indicated that the activation of protein kinase C (PKC) and increased diacylglycerol (DAG) levels initiated by hyperglycemia are associated with many vascular abnormalities in retinal, renal, and cardiovascular tissues (Koya, D. and King, G. Diabetes 47:859-866 (1998)). Among the various PKC isoforms, the beta- and delta-isoforms appear to be activated preferentially in the vasculatures of diabetic animals (Inoguchi et al. Proc. Natl Acad Sci USA 89:11059-11063 (1992); Ishii et al. Science 272: 728-731 (1996)), although other PKC isoforms are also increased in the renal glomeruli and retina. The glucose-induced activation of PKC has been shown to increase the production of extracellular matrix and cytokines; to enhance contractility, permeability, and vascular cell proliferation; to induce the activation of cytosolic phospholipase A2; and to inhibit Na+-K+-ATPase. The synthesis and characterization of a specific inhibitor for PKC-beta isoforms has confirmed the role of PKC activation in mediating hyperglycemic effects on vascular cells, and provided in vivo evidence that PKC activation could be responsible for abnormal retinal and renal hemodynamics in diabetic animals (Ishii et al. Science 272: 728-731 (1996)). Transgenic mice overexpressing PKC-beta isoform in the myocardium developed cardiac hypertrophy and failure, further supporting the hypothesis that PKC-beta isoform activation can cause vascular dysfunctions (Bowman et al. J Clin Invest. 100(9): 2189-2195 (1997)).

In another embodiment, inhibitors of PKC are used in combination with an HMG CoA reductase inhibitor and/or a pFox inhbitor for the treatment of patients with diabetes or metabolic syndrome and endothelial dysfunction. PKC inhibitors, and methods for their preparation are readily available in the art. For example, different kinds of PKC inhibitors and their preparation are described in U.S. Pat. Nos. 5,621,101; 5,621,098; 5,616,577; 5,578,590; 5,545,636; 5,491,242; 5,488,167; 5,481,003; 5,461,146; 5,270,310; 5,216,014; 5,204,370; 5,141,957; 4,990,519; and 4,937,232. Examples of PKC inhibitors include AG 490, PD98059, PKC-alpha/beta pseudosubstrate peptide, staurosporine Ro-31-7549, Ro-31-8220, Ro-31-8425, Ro-32-0432, H-7, sangivamycin; calphostin C, safingol, D-erythro-sphingosine, chelerythrine chloride, melittin; dequalinium chloride, Go6976, Go6983; Go7874, polymyxin B sulfate; cardiotoxin, ellagic acid, HBDDE, 1-O-Hexadecyl-2-O-methyl-rac-glycerol, hypercin, K-252, NGIC-J, phloretin, piceatannol, tamoxifen citrate, flavopiridol, and bryostatin 1. In a preferred embodiment, the inhibitor selectively inhibits the beta- and/or delta-isoforms of PKC. Suitable small molecule PKC-beta inhibitors include LY333531 (developed by Eli Lilly as Ruboxistaurin™). Recent data with this compound from a study of patients receiving 32 mg/day, suggests that ruboxistaurin may have the potential to decrease the progression of diabetic macular edema to involve the center of the macula.

### F. Acetyl-CoA Carboxylase Inhibitors

Acetyl-CoA carboxylase (ACC) catalyzes the rate-limiting reaction in fatty acid biosynthesis (Kim, K. H. (1997) Annu. Rev. Nutr. 17, 77-99; Munday, M. R., and Hemingway, C. J. (1999) Adv. Enzyme Reg. 39, 205-234). In animals, including humans, there are two isoforms of acetyl-CoA carboxylase expressed in most cells, ACCI (Mᵣ about. 265,000) and ACC2 (Mᵣ about 280,000), which are encoded by two separate genes and display distinct tissue distribution. Both ACC1 and ACC2 produce malonyl-CoA, which inhibits mitochondrial fatty acid oxidation through feedback inhibition of carnitine palmitoyltransferase 1 (CPT-1) )McGarry, J. D., Woeltje, K. F., Kuwajima, M., and Foster, D. W. (1989) Diabetes Metabol. Revs. 5, 271-284 and McGarry, J. D., and Brown, N. F. (1997) Eur. J Biochem. 244, 1-14), and therefore plays key roles both in controlling the switch between carbohydrate and fatty acid utilization in liver and skeletal muscle and also in regulating insulin sensitivity in the liver, skeletal muscle, and adipose tissue (McGarry, J. D., Woeltje, K. F., Kuwajima, M., and Foster, D. W. (1989) Diabetes Metabol. Revs. 5, 271-284; McGarry, J. D., and Brown, N. F. (1997) Eur. J Biochem. 244, 1-14). Malonyl-CoA may also play an important regulatory role in controlling insulin secretion from the pancreas (Chen, S., Ogawa, A., Ohneda, M., Unger, R. H., Foster, D. W., and J. D. McGarry (1994) Diabetes 43, 878-883).

Thus, in addition to inhibition of fatty acid synthesis, reduction in malonyl-CoA levels through ACC inhibition may provide a mechanism for increasing fatty acid utilization that may reduce TG-rich lipoprotein secretion (very low density lipoprotein) by the liver, alter insulin secretion by the pancreas, and improve insulin sensitivity in liver, skeletal muscle, and adipose tissue. Additionally, by increasing fatty acid utilization and by preventing increases in de novo fatty acid synthesis, chronic administration of an ACC inhibitor may also deplete liver and adipose tissue TG stores in obese subjects consuming a low fat diet, leading to selective loss of body fat.

Therefore, an ACC inhibitor can be used to effectively and simultaneously treat the multiple risk factors associated with metabolic syndrome and could have a significant impact on the prevention and treatment of the cardiovascular morbidity and mortality associated with obesity, hypertension, diabetes, and atherosclerosis. In another embodiment, ACC inhibitors are used in combination with an HMG CoA reductase inhibitor and/or a pFox inhibitor for the treatment of patients with diabetes or metabolic syndrome and endothelial dysfunction. Examples of suitable acetyl-CoA carboxylase inhibitors are described in U.S. Pat. Nos. 6,734,337 and 6,485,941 and in Harwood et al. J. Biol. Chem., Vol. 278, Issue 39, 37099-37111 (2003). These include compounds such as the isozyme-nonselective ACC inhibitors CP-640186 and CP-610431.

### G. Rho-Kinase Inhibitors

Increased activity of Rho-kinase causes hypercontraction of vascular smooth muscle and has been implicated as playing a pathogenetic role in divergent cardiovascular diseases such as coronary artery spasm. Vasospastic angina is a form of angina caused by coronary artery spasm. Compounds which inhibit rho-kinase can be used to treat this form of angina. Suitable compounds include the selective rho-kinase inhibitor fasudil.

### H. Formulations

Preferably, the compounds are orally administered. For oral administration, the compounds, particularly their acid addition salts, are formed into tablets, granules, powders or capsules containing suitable amounts of granules or powders by a conventional method together with usual drug additives. Oral formulations containing the active compounds may be in any conventionally used oral form, including tablets, capsules, buccal forms, troches, lozenges and oral liquids, suspensions or solutions. Oral formulations may utilize standard delay or time release formulations to alter the absorption of the active compound(s).

Formulation of drugs is discussed in, for example, Hoover, John E., Remington's Pharmaceutical Sciences, Mack Publishing Co., Easton, Pa. (1975), and Liberman, H. A. and Lachman, L., Eds., Pharmaceutical Dosage Forms, Marcel Decker, New York, N.Y. (1980). The active compounds (or pharmaceutically acceptable salts thereof) may be administered in the form of a pharmaceutical composition wherein the active compound(s) is in admixture or mixture with one or more pharmaceutically acceptable carriers, excipients or diluents. Pharmaceutical compositions may be formulated in conventional manner using one or more physiologically acceptable carriers comprising excipients and auxiliaries which facilitate processing of the active compounds into preparations which can be used pharmaceutically. Proper formulation is dependent upon the route of administration chosen.

### 1. Carriers, Excipients, and Diluents

Optional pharmaceutically acceptable excipients present in the drug-containing tablets, beads, granules or particles include, but are not limited to, diluents, binders, lubricants, disintegrants, colorants, stabilizers, and surfactants.

Diluents, also referred to as “fillers,” are typically necessary to increase the bulk of a solid dosage form so that a practical size is provided for compression of tablets or formation of beads and granules. Suitable diluents include, but are not limited to, dicalcium phosphate dihydrate, calcium sulfate, lactose, sucrose, mannitol, sorbitol, cellulose, microcrystalline cellulose, kaolin, sodium chloride, dry starch, hydrolyzed starches, pregelatinized starch, silicone dioxide, titanium oxide, magnesium aluminum silicate and powdered sugar.

Binders are used to impart cohesive qualities to a solid dosage formulation, and thus ensure that a tablet or bead or granule remains intact after the formation of the dosage forms. Suitable binder materials include, but are not limited to, starch, pregelatinized starch, gelatin, sugars (including sucrose, glucose, dextrose, lactose and sorbitol), polyethylene glycol, waxes, natural and synthetic gums such as acacia, tragacanth, sodium alginate, cellulose, including hydroxypropylmethylcellulose, hydroxypropylcellulose, ethylcellulose, and veegum, and synthetic polymers such as acrylic acid and methacrylic acid copolymers, methacrylic acid copolymers, methyl methacrylate copolymers, aminoalkyl methacrylate copolymers, polyacrylic acid/polymethacrylic acid and polyvinylpyrrolidone.

Lubricants are used to facilitate tablet manufacture. Examples of suitable lubricants include, but are not limited to, magnesium stearate, calcium stearate, stearic acid, glycerol behenate, polyethylene glycol, talc, and mineral oil.

Disintegrants are used to facilitate dosage form disintegration or “breakup” after administration, and generally include, but are not limited to, starch, sodium starch glycolate, sodium carboxymethyl starch, sodium carboxymethylcellulose, hydroxypropyl cellulose, pregelatinized starch, clays, cellulose, alginine, gums or cross linked polymers, such as cross-linked PVP (Polyplasdone XL from GAF Chemical Corp).

Stabilizers are used to inhibit or retard drug decomposition reactions which include, by way of example, oxidative reactions.

Surfactants may be anionic, cationic, amphoteric or nonionic surface active agents. Suitable anionic surfactants include, but are not limited to, those containing carboxylate, sulfonate and sulfate ions. Examples of anionic surfactants include sodium, potassium, ammonium of long chain alkyl sulfonates and alkyl aryl sulfonates such as sodium dodecylbenzene sulfonate; dialkyl sodium sulfosuccinates, such as sodium dodecylbenzene sulfonate; dialkyl sodium sulfosuccinates, such as sodium bis-(2-ethylthioxyl)-sulfosuccinate; and alkyl sulfates such as sodium lauryl sulfate. Cationic surfactants include, but are not limited to, quaternary ammonium compounds such as benzalkonium chloride, benzethonium chloride, cetrimonium bromide, stearyl dimethylbenzyl ammonium chloride, polyoxyethylene and coconut amine. Examples of nonionic surfactants include ethylene glycol monostearate, propylene glycol myristate, glyceryl monostearate, glyceryl stearate, polyglyceryl-4-oleate, sorbitan acylate, sucrose acylate, PEG-150 laurate, PEG-400 monolaurate, polyoxyethylene monolaurate, polysorbates, polyoxyethylene octylphenylether, PEG-1000 cetyl ether, polyoxyethylene tridecyl ether, polypropylene glycol butyl ether, Poloxamer® 401, stearoyl monoisopropanolamide, and polyoxyethylene hydrogenated tallow amide. Examples of amphoteric surfactants include sodium N-dodecyl-.beta.-alanine, sodium N-lauryl-.beta.-iminodipropionate, myristoamphoacetate, lauryl betaine and lauryl sulfobetaine.

If desired, the tablets, beads, granules, or particles may also contain minor amount of nontoxic auxiliary substances such as wetting or emulsifying agents, dyes, pH buffering agents, or preservatives.

Blending or copolymerization sufficient to provide a certain amount of hydrophilic character can be useful to improve wettability of the materials. For example, about 5% to about 20% of monomers may be hydrophilic monomers. Hydrophilic polymers such as hydroxylpropylcellulose (HPC), hydroxpropylmethylcellulose (HPMC), carboxymethylcellulose (CMC) are commonly used for this purpose. Also suitable are hydrophobic polymers such as polyesters and polyimides. It is known to those skilled in the art that these polymers may be blended with polyanhydrides to achieve compositions with different drug release profiles and mechanical strengths. Preferably, the polymers are bioerodable, with preferred molecular weights ranging from 1000 to 15,000 kDa, and most preferably 2000 to 5000 Da.

The compounds may be complexed with other agents as part of their being pharmaceutically formulated. The pharmaceutical compositions may take the form of, for example, tablets or capsules prepared by conventional means with pharmaceutically acceptable excipients such as binding agents (e.g., acacia, methylcellulose, sodium carboxymethylcellulose, polyvinylpyrrolidone (Povidone), hydroxypropyl methylcellulose, sucrose, starch, and ethylcellulose); fillers (e.g., corn starch, gelatin, lactose, acacia, sucrose, microcrystalline cellulose, kaolin, mannitol, dicalcium phosphate, calcium carbonate, sodium chloride, or alginic acid); lubricants (e.g. magnesium stearates, stearic acid, silicone fluid, talc, waxes, oils, and colloidal silica); and disintegrators (e.g. micro-crystalline cellulose, corn starch, sodium starch glycolate and alginic acid. If water-soluble, such formulated complex then may be formulated in an appropriate buffer, for example, phosphate buffered saline or other physiologically compatible solutions. Alternatively, if the resulting complex has poor solubility in aqueous solvents, then it may be formulated with a non-ionic surfactant such as TWEEN™, or polyethylene glycol. Thus, the compounds and their physiologically acceptable solvates may be formulated for administration.

Liquid formulations for oral administration prepared in water or other aqueous vehicles may contain various suspending agents such as methylcellulose, alginates, tragacanth, pectin, kelgin, carrageenan, acacia, polyvinylpyrrolidone, and polyvinyl alcohol. The liquid formulations may also include solutions, emulsions, syrups and elixirs containing, together with the active compound(s), wetting agents, sweeteners, and coloring and flavoring agents. Various liquid and powder formulations can be prepared by conventional methods for inhalation by the patient.

### 2. Modified Release Formulations

Delayed release and extended release compositions can be prepared. The delayed release/extended release pharmaceutical compositions can be obtained by complexing drug with a pharmaceutically acceptable ion-exchange resin and coating such complexes. The formulations are coated with a substance that will act as a barrier to control the diffusion of the drug from its core complex into the gastrointestinal fluids. Optionally, the formulation is coated with a film of a polymer which is insoluble in the acid environment of the stomach, and soluble in the basic environment of lower GI tract in order to obtain a final dosage form that releases less than 10% of the drug dose within the stomach. Examples of suitable coating materials include, but are not limited to, cellulose polymers such as cellulose acetate phthalate, hydroxypropyl cellulose, hydroxypropyl methylcellulose, hydroxypropyl methylcellulose phthalate and hydroxypropyl methylcellulose acetate succinate; polyvinyl acetate phthalate, acrylic acid polymers and copolymers, and methacrylic resins that are commercially available under the trade name EUDRAGIT® (Roth Pharma, Westerstadt, Germany), zein, shellac, and polysaccharides.

Examples of rate controlling polymers that may be used in the dosage form are hydroxypropylmethylcellulose (HPMC) with viscosities of either 5, 50, 100 or 4000 cps or blends of the different viscosities, ethylcellulose, methylmethacrylates, such as Eudragit RS100, Eudragit RL100, Eudragit NE 30D (supplied by Rohm America). Gastrosoluble polymers, such as Eudragit E100 or enteric polymers such as Eudragit L100-55D, L100 and S100 may be blended with rate controlling polymers to achieve pH dependent release kinetics. Other hydrophilic polymers such as alginate, polyethylene oxide, carboxymethylcellulose, and hydroxyethylcellulose may be used as rate controlling polymers.

Additionally, the coating material may contain conventional carriers such as plasticizers, pigments, colorants, glidants, stabilization agents, pore formers and surfactants.

The drugs may optionally be encapsulated or molecularly dispersed in polymers to reduce particle size and increase dissolution. The polymers may include polyesters such as poly (lactic acid) or P(LA), polycaprylactone, polylactide-coglycolide or P(LGA), poly hydroxybutyrate poly β-malic acid); polyanhydrides such as poly (adipic)anhydride or P(AA), poly (fumaric-co-sebacic) anhydride or P(FA:SA), poly (sebacic) anhydride or P(SA); cellulosic polymers such as ethylcellulose, cellulose acetate, cellulose acetate phthalate, etc; acrylate and methacrylate polymers such as Eudragit RS 100, RL 100, E100 PO, L100-55, L100, S100 (distributed by Rohm America) or other polymers commonly used for encapsulation for pharmaceutical purposes and known to those skilled in the art.

Suppository formulations may be made from traditional materials, including cocoa butter, with or without the addition of waxes to alter the suppository's melting point, and glycerin. Water soluble suppository bases, such as polyethylene glycols of various molecular weights, may also be used.

Alternatively, the compound may be administered in liposomes or microspheres (or microparticles). Methods for preparing liposomes and microspheres for administration to a patient are known to those skilled in the art. U.S. Pat. No. 4,789,734 describe methods for encapsulating biological materials in liposomes. Essentially, the material is dissolved in an aqueous solution, the appropriate phospholipids and lipids added, along with surfactants if required, and the material dialyzed or sonicated, as necessary. A review of known methods is by G. Gregoriadis, Chapter 14. “Liposomes”, Drug Carriers in Biology and Medicine pp. 287-341 (Academic Press, 1979). Microspheres formed of polymers or proteins are well known to those skilled in the art, and can be tailored for passage through the gastrointestinal tract directly into the bloodstream. Alternatively, the compound can be incorporated and the microspheres, or composite of microspheres, implanted for slow release over a period of time, ranging from days to months. See, for example, U.S. Pat. Nos. 4,906,474, 4,925,673, and 3,625,214.

### II. CONDITIONS TO BE TREATED

The combination of an HMG CoA reductase inhibitor, such as a statin (e.g., “simvastatin”), in combination with a pFox inhibitor, such as trimetazidine (“Simetazidine”), is beneficial for treatment of acute coronary syndrome (ACS) and chronic angina, particularly in diabetics. HDL activates eNOS and both simvastatin and atorvastatin increase HDL, with atorvastatin more than simvastatin. Trimetazidine also raises HDL, and may be therapeutic by virtue of being an agonist of eNOS, as well as being a pFOX inhibitor. Accordingly, part of the benefit of the treatment of acute coronary syndrome is the lowering of CRP.

This combination is useful for the treatment of these conditions in diabetic and non-diabetic patients. In patients with diabetes, especially Type II diabetes, the addition of one or more oral hypoglycemic compound to the pFox inhibitor and HMG CoA reductase inhibitor is particularly advantageous to control glucose levels. The combinations can also be used to treat patients who cannot take beta blockers, such as those suffering from sick sinus syndrome (slow heart rhythms) and other conduction system disturbances as well as those patients suffering from asthma and chronic obstructive lung diseases accompanied by bronchospasm.

Diabetes has been commonly characterized as a disease of impaired skeletal muscle glucose uptake, however, diabetes also adversely affects hepatic, muscle, adipose, and vascular function. It is this last effect that may represent the greatest mortality hazard. Diabetes creates an environment adverse to vascular function through a wide variety of dysmetabolic assaults.

There are two clinical forms of diabetes, each with a different pathogenesis: type 1, insulin dependent diabetes mellitus and type 2, non-insulin dependent diabetes mellitus. The latter represents 90% of all diabetics. Type I diabetes (IDDM) is the result of an absolute deficiency of insulin, the hormone which regulates glucose utilization. Type II, noninsulin dependent diabetes mellitus (NIDDM) is due to a profound resistance to insulin stimulating or regulatory effect on glucose and lipid metabolism in the main insulin-sensitive tissues, muscle, liver and adipose tissue. This resistance to insulin responsiveness results in insufficient insulin activation of glucose uptake, oxidation and storage in muscle and inadequate insulin repression of lipolysis in adipose tissue and of glucose production and secretion in liver.

Over the past decade, type II diabetes mellitus has reached epidemic levels in the United States and world wide. Half of all patients with type II diabetes have evidence of coronary artery disease and the vast majority of diabetes-related hospital admissions are for atherosclerotic vascular disease. Diabetes increases the frequency of stroke, heart attack, and amputation 2- to 4-fold putting these patients at risk.

The combination therapy is also useful in the treatment and/or prevention of metabolic syndrome, a collection of major and emerging cardiovascular risk factors that stem from underlying insulin resistance. Metabolic syndrome is a common precursor to both atherosclerotic vascular disease (ASCVD) and type II diabetes. Metabolic syndrome likely develops from obesity, physical inactivity, and atherogenic diet, although a genetic predisposition may contribute. These factors lead to insulin resistance, which, in turn, contribute to a typical set of major and emerging risk factors: abdominal obesity; elevated blood pressure; atherogenic dyslipidemia (high triglycerides, low HDL, and small, dense LDL); impaired fasting glucose or glucose intolerance; proinflammatory state; and prothrombotic state. By definition 3 or more of these risk factors constitutes the metabolic syndrome.

The combination therapy is also useful in the treatment and/or prevention of chronic heart failure (CHF) and peripheral arterial disease (PAD). The patients to be treated are those characterized by an inadequate response to current cholesterol lowering and medications for hypotension. Trimetazidine is particularly preferred because it does not prolong QT interval and is very effective at controlling angina, especially in patients with marginally low blood pressure. Insofar as QT_{c} is part of the natural history of unstable angina and myocardial infarction and predisposes to sudden death, it is desirable to use an agent that does not further aggravate the risk associated with these conditions.

Another preferred condition for treatment is chronic intractable (inoperable) angina. This condition involves the smaller vessels supplying blood to the myocardium and is sometimes called microvascular angiopathy. It is typically seen in individuals with diabetes. It is generally not responsive to treatment by mechanical revascularization methods such as angioplasty and stenting or bypass surgery because of the small size of the vessels involved.

Acute coronary syndromes involve blockages in blood flow to heart muscle. If the blockage lasts long enough, an area of heart muscle dies, a condition commonly known as a heart attack or myocardial infarction. The goal of treatment for acute coronary syndromes is to keep heart muscle alive and prevent bad outcomes such as heart attack or death.

The combination involves the two mechanisms of increasing endothelium derived nitric oxide production (“EDNO”) and pFox inhibition. The goal of the treatment is to reduce ischemia and reperfusion injury, promote ischemic preconditioning and reduce tissue damage (e.g., substantially preventing tissue damage and/or inducing tissue protection) resulting from ischemia using different mechanisms to enhance efficacy. The combination of a nitric oxide generator and a metabolic modulator/pFoxᵢ that results in a non-hemodynamic synergistic interaction improves oxygen ultilization by the myocardium. Preferred ischemic tissues that may be treated include, in addition to cardiovascular tissues, brain, liver, kidney, lung, gut, skeletal muscle, spleen, pancreas, nerve, spinal cord, retinal tissue, peripheral vasculature, intestinal and genitourinary tissue.

An effective amount of the formulation is that amount which provides relief of angina, claudication, silent ischemia and/or their equivalents. Reductions in clinical outcomes and endpoints include, but are not limited to, total mortality and cardiovascular mortality, need for procedures such as stenting and bypass operations and hospitalizations.

The statin is preferably given in a dose of between 5 and 80 mg/day in two or three separate doses. In a preferred embodiment, the pFox inhibitor is administered in a dosage of between 5 and 1000 mg/day, more preferably between 10 and 100 mg/day, most preferably between 60 and 90 mg/day. In a more preferred embodiment, the pFox inhibitor trimetazidine is administered in a sustained or extended dosage formulation at a dose of 45 mg two times a day or in an immediate release formulation at a dose of 20 mg three times a day.

Examples of suitable combinations include 13.33 mg simvastatin with 20 mg of trimetazidine given three times a day; 20 mg simvastatin with 45 mg of extended release trimetazidine given twice daily; 26.66 mg atorvastatin with 20 mg of trimetazidine given three times a day; 40 mg atorvastain with 45 mg of extended release trimetazidine given twice a day; 10 mg of simvastatin with 250 mg of mildronate given twice daily (two tablets); 20 mg simvastatin with 250 mg mildronate one daily (one tablet); and 20 mg atorvastatin with 250 mg mildronate given twice daily (1-2 tablets). Statin-mildronate combinations can also be administered intravenously, which in combination with a statin, for example, pravastatin i.v., may be useful for treatment of acute coronary syndrome.

If the statin is simvastatin, the most preferred administration regime is 20 mg of simvastatin combined in a single tablet or capsule with 45 mg of trimetazidine extended release and dosed twice daily. If the statin is atorvastatin, the most preferred regime is 40 mg of atorvastatin combined in a single tablet or capsule with 45 mg of trimetazidine extended release and dosed twice daily.

If an oral hypoglycemic is added to the combination of pFox inhibitor and HMG CoA reductase inhibitor, preferred drugs and doses include glimepiride, administered in a dose of from 0.5 to 4 mg/day; glipizide, administered in a dose of from 5 to 20 mg/day; rosaglitazone, administered in a dose of from 100 mg to 600 mg/day; metformin, administered in a dose of from 250 to 2000 mg/day; a combination of glipizide and metformin administered in a dose from 2.5 mg/day glipazide/250 mg/day metformin to 10 mg/day glipazide/2,000 mg/day metformin; a combination of glyburide and metformin administered in a dose of from 1.25 mg/day glyburide/250 mg/day metformin to 10 mg/day glyburide/2,000 mg/day metformin; and a combination of rosaglitazone and metformin administered in a dose of from 1 mg/day rosiglitazone/250 mg/day metformin to 8 mg/day rosiglitazone/2,000 mg/day metformin. In another embodiment, a combination of a nitric oxide generator and a pFox inhibitor which results in a non-hemodynamic interaction is administered to improve oxygen utilization by the myocardium.

One or more of the components may be provided in a sustained or immediate release formulation, where one is sustained and the other is immediate release, or all may be sustained, delayed or immediate release formulations. The formulations may be packaged together or separately in combination with instructions for administration of the desired combinations.

It is understood that the described methods are not limited to the particular methodology, protocols, and reagents described as these may vary. It is also to be understood that the terminology used herein is for the purpose of describing particular embodiments only, and is not intended to limit the scope of the present invention which will be limited only by the appended claims. Unless defined otherwise, all technical and scientific terms used herein have the same meanings as commonly understood by one of skill in the art to which the described invention belongs.

Those skilled in the art will recognize, or be able to ascertain using no more than routine experimentation, many equivalents to the specific embodiments of the invention described herein. Such equivalents are intended to be encompassed by the following claims.

I claim:

1. A method of treating a patient in need thereof for endothelial dysfunction comprising administering an effective amount to alleviate at least one symptom of endothelial dysfunction of a combination of
a HMG CoA reductase inhibitor, and
a partial fatty acid oxidation (“pFox”) inhibitor.

2. The method of claim 1 further comprising providing a nitric oxide agonist, nitric oxide generator or an upregulator of nitric oxide synthase (“eNOS agonist”), or selecting a HMG CoA reductase inhibitor or pFox inhibitor having nitric oxide agonist, nitric oxide generating or nitric oxide synthase upregulating activity.

3. The method of claim 1 wherein the HMG CoA reductase inhibitor is a statin.

4. The method of claim 1 wherein the pFox inhibitor is selected from the group consisting of a ranolazine, trimetazidine, perhexiline maleate, and mildronate.

5. The method of claim 1 wherein the HMG CoA reductase inhibitor is a statin in a dose of between 5 and 80 mg/day in between one and three separate doses, and the pFox inhibitor is trimetazidine administered in a dose of between 5 and 1000 mg/day, more preferably between 10 and 100 mg/day, most preferably between 60 and 90 mg/day.

6. The method of claim 5 wherein the pFox inhibitor is trimetazidine administered in a sustained or extended dosage formulation at a dose of 45 mg two times a day or in an immediate release formulation at a dose of 20 mg three times a day.

7. The method of claim 1 further comprising administering the combination of an nitric oxide agonist, nitric oxide generator or upregulator of nitric oxide synthase, angiotensin receptor blocker, and ACE inhibitor.

8. The method of claim 1 further comprising administering the combination of an inhibitor of inducible nitric oxide synthase which causes constitutive NO synthase to activate.

9. The method of claim 1 further comprising administering a nitric oxide agonist or an upregulator of nitric oxide synthase.

10. The method of claim 2 wherein the agonist or upregulator is selected from the group consisting of angiotensin II receptor blockers (ARB's), and angiotensin converting enzyme (ACE) inhibitors, endothelial nitric oxide synthase agonists, peroxisome proliferator-activated receptor activators, and cilostazol.

11. The method of claim 1 further comprising administering one or more oral hypoglycemic compounds selected from the group consisting of biguanides, insulin sensitizers, such as thiazolidinediones, α-glucosidase inhibitors, insulin secretagogues, and dipeptidyl peptidase IV inhibitors, protein kinase C (PKC) inhibitors, or acetyl-CoA carboxylase inhibitors in combination with the HMG CoA reductase inhibitor and pFox inhibitor, to control glucose levels and treat endothelial dysfunction

12. The method of claim 1 wherein the combination is a package with separate dosage forms for the HMG CoA reductase inhibitor and the partial fatty acid oxidation (“pFox”) inhibitor, and instructions for administration of each to produce a combined effect.

13. The method of claim 1 wherein the partial fatty acid oxidation (“pFox”) inhibitor is in a sustained release formulation and the HMG CoA reductase inhibitor is in an immediate release formulation.

14. The method of claim 1 wherein the partial fatty acid oxidation (“pFox”) inhibitor or the HMG CoA reductase inhibitor is in an immediate release formulation and the other is in a delayed or sustained release formulation.

15. The method of claim 1 wherein both the partial fatty acid oxidation (“pFox”) inhibitor and HMG Co A reductase inhibitor are immediate release or sustained release formulations.

16. The method of claim 1 wherein the combination is administered in a combination dosage effective to provide relief of angina, claudication, silent ischemia, or their equivalents, as measured by reductions in clinical outcomes, total mortality, cardiovascular mortality, reduction in stenting, bypass operations or hospitalizations.

17. The method of claim 1 for treatment of acute coronary syndrome (ACS).

18. The method of claim 1 for treatment of myocardial infarction (MI), stroke or cerebrovascular attack (CVA).

19. The method of claim 1 for treatment of chronic angina, unstable angina, microvascular angina due to left ventricular hypertrophy, or microvascular angiopathy.

20. The method of claim 1 for treatment of silent myocardial ischemia.

21. The method of claim 1 wherein the patient has diabetes.

22. The method of claim 1 for the treatment and/or prevention of chronic heart failure (CHF).

23. The method of claim 1 for treatment of peripheral arterial disease (PAD) and claudication.

24. The method of claim 1 for treatment of transient ischemic attacks (TIAs).

25. The method of claim 1 for treatment of silent ischemia, ischemia, reperfusion injury, or inducing ischemic preconditioning.

26. The method of claim 1 for treatment of coronary and myocardial insufficiency, mesenteric ischemia, pulmonary hypertension, and erectile dysfunction (ED).

27. The method of claim 1 wherein a combination of a nitric oxide generator and a pFox inhibitor which results in a non-hemodynamic interaction is administered to improve oxygen utilization by the myocardium.

28. A method of treating a patient in need thereof for metabolic syndrome or diabetes and endothelial dysfunction comprising administering a combination of two or more compounds selected from the group consisting of an HMG CoA reductase inhibitor, a partial fatty acid oxidation (“pFox”) inhibitor, one or more oral hypoglycemics, a protein kinase C inhibitor, and an acetyl-CoA carboxylase inhibitor.

29. The method of claim 28 wherein the combination comprises an HMG CoA reductase inhibitor, a partial fatty acid oxidation (“pFox”) inhibitor, and one or more oral hypoglycemics.

30. The method of claim 28 wherein the combination comprises an HMG CoA reductase inhibitor and a compound selected from the group consisting of a protein kinase C inhibitor and an acetyl-CoA carboxylase inhibitor.

31. The method of claim 28 wherein the combination comprises a partial fatty acid oxidation inhibitor and a compound selected from the group consisting of a protein kinase C inhibitor and an acetyl-CoA carboxylase inhibitor.

32. The method of claim 28 comprising a HMG CoA reductase inhibitor wherein the HMG CoA reductase inhibitor is a statin.

33. The method of claim 28 comprising a pFOX inhibitor, wherein the pFox inhibitor is selected from the group consisting of a ranolazine and trimetazidine.

34. The method of claim 28 comprising an oral hypoglycemic, wherein the oral hypoglycemic is selected from the group of compounds consisting of biguanides, thiazolidinediones, alpha-glucosidase inhibitors, insulin secretagogues, and dipeptidyl peptidase IV inhibitors.

35. The method of claim 34 wherein the biguanide is metformin; the thiazolidinedione is selected from the group consisting of troglitazone, rosiglitazone, pioglitazone, ciglitazone, englitazone, and R483; the alpha-glucosidase inhibitor is selected from the group consisting of acarbose, adiposine, voglibose, miglitol, emiglitate, camiglibose, tendamistate, trestatin, pradimicin-Q and salbostatin; the insulin secretagogue is selected from the group consisting of chlorpropamide, tolazimide, tolbutamide, gliclazide, glipizide, glipizide GITS, glyburide, micronized glyburide, glimepiride, AG-EE 623 ZW, nateglinide, meglitinides, and repaglinide; the dipeptidyl peptidase IV inhibitor is selected from the group consisting of L-threo-isoleucyl pyrrolidide, L-allo-isoleucyl thiazolidide, L-allo-isoleucyl pyrrolidide, valine pyrrolidide, NVP-DPP728A, LAF-237, TSL-225, FE-999011, GW-229A, 815541, MK-431 and PT-100; and the protein kinase C inhibitor is LY333531.

36. The method of claim 28 wherein the combination is a package with separate dosage forms for two or more of the compounds.

37. The method of claim 28 comprising a pFox inhibitor, HMG Co A reductase inhibitor and one or more oral hypoglycemics in immediate release or sustained release formulations.

38. The method of claim 28 comprising a HMG CoA reductase inhibitor which is a statin in a dose of from 5 to 80 mg/day in between one and three separate doses, a pFox inhibitor trimetazidine which is administered in a dose of from 5 to 1000 mg/day, and a oral hypoglycemic which is selected from the group consisting of glimepiride, administered in a dose of from 0.5 to 4 mg/day; glipizide, administered in a dose of from 5 to 20 mg/day; rosaglitazone, administered in a dose of from 100 mg to 600 mg/day; metformin, administered in a dose of from 250 to 2000 mg/day; a combination of glipizide and metformin administered in a dose from 2.5 mg/day glipazide/250 mg/day metformin to 10 mg/day glipazide/2,000 mg/day metformin; a combination of glyburide and metformin administered in a dose of from 1.25 mg/day glyburide/250 mg/day metformin to 10 mg/day glyburide/2,000 mg/day metformin; and a combination of rosaglitazone and metformin administered in a dose of from 1 mg/day rosiglitazone/250 mg/day metformin to 8 mg/day rosiglitazone/2,000 mg/day metformin.

39. The method of claim 28 wherein the diabetes is type II diabetes.

40. The method of claim 28 wherein the patient has coronary artery disease.

41. The method of claim 28 wherein the patient has atherosclerotic vascular disease.

42. The method of claim 28 wherein the patient has three or more risk factors for metabolic syndrome selected from the group consisting of abdominal obesity; elevated blood pressure; atherogenic dyslipidemia (high triglycerides, low HDL, and small, dense LDL); impaired fasting glucose or glucose intolerance; proinflammatory state; and prothrombotic state.

43. The method of claim 28 wherein the patient has chronic angina, unstable angina, microvascular angina due to left ventricular hypertrophy, or microvascular angiopathy.

44. The method of claim 28 wherein the patient has congestive heart failure (CHF).

45. The method of claim 28 wherein the patient has peripheral arterial disease (PAD) and claudication.

46. A formulation for use in the method of claim 1.

47. A formulation for use in the method of claim 28.
